(19) United States
(12) Patent Application Publication
(10) Pub. No.: US 2007/0179426 A1
(43) Pub. Date: Aug. 2, 2007

(54) Interfacial stent and method of maintaining patency of surgical fenestrations

(76) Inventor: Nathan R. Selden, Portland, OR (US)

Correspondence Address: KLARQUIST SPARKMAN, LLP, 121 SW SALMON STREET, SUITE 1600, PORTLAND, OR, 97204, US

(21) Appl. No.: 11/596,270

(22) PCT Filed: May 9, 2005
(86) PCT No.: PCT/US05/16350

§ 371 Date: Nov. 10, 2006

Related U.S. Application Data
(60) Provisional application No. 60/570,178, filed on May 11, 2004.

(51) Int. Cl. A61F 2/04; A61F 2/84; A61F 2/90

(52) U.S. Cl. 604/8; 623/1.12; 623/23.7

(57) ABSTRACT

A method according to one embodiment for maintaining patency of an opening inside the human body comprises introducing a radially self-expanding hollow stent into the opening through an endoscope that radially compresses the stent, wherein the stent has enlarged ends and a reduced intermediate portion. The stent is introduced into the opening such that its intermediate portion extends through the opening and the enlarged ends are positioned outside of the opening. Once deployed, the stent expands such that the enlarged ends of the stent abut against opposing faces of the opening to resist dislodgement of the stent from the opening after expansion. The stent is preferably biodegradable, such that it is eliminated from the surgical site over a period of weeks to months, by which time the patency of the opening is more assured. The method can be used in combination with, for example, an endoscopic surgical method such as endoscopic third ventriculostomy for treating hydrocephalus of a brain.

### CROSS-REFERENCE TO RELATED APPLICATION

The present application claims the benefit of U.S. Provisional Application No. 60/570,178, filed May 11, 2004, which is incorporated herein by reference.

### STATEMENT OF GOVERNMENT SUPPORT

This invention has not been developed with any government support.

### BACKGROUND OF THE INVENTION

The present disclosure relates to implantable stents used inside the human body for medical purposes.

The human body includes many anatomical pathways through which body fluids, such as blood or cerebrospinal fluid (CSF), must pass to maintain proper biological function. Examples of such pathways are elongated blood vessels (such as the coronary arteries) and other extended passageways that define a lumen (such as the aqueduct of Sylvius in the ventricular system of the brain). Obstructions of biological lumens can cause serious medical problems, such as tissue ischemia secondary to occlusion of an artery, or hydrocephalus caused by disruption of the flow of CSF through the ventricular system.

In the case of obstruction of an elongated vessel, such as stenosis of a blood vessel in the cardiovascular system, implantable intra-luminal stents have been used to maintain patency of the vessel lumen. Intravascular stents are commonly placed in an atherosclerotic coronary artery to reestablish perfusion to ischemic cardiac tissue. Coronary stents are introduced along a catheter to a site of occlusion during an angioplasty procedure. The stents, which are typically tubular in shape, may be expanded mechanically or by the introduction of pressurized air into a balloon placed in the lumen of the stent. Coronary stents are not usually designed to be biodegradable, because they are intended to provide long-term mechanical support to maintain patency of the vessel lumen.

In addition to using stents, surgeons often employ other operative techniques to reestablish normal flow of fluids through biological pathways in the body. For example, an artificial opening such as a surgical fenestration may be created in a biological interface (such as a membrane or other tissue barrier) to either reopen a natural pathway or to create a new pathway for therapeutic purposes. Endoscopic surgery procedures may involve fenestration of a biological interface inside the body, in which a small opening is surgically created to establish or facilitate communication of such fluids as blood, bile, aqueous humor or cerebrospinal fluid (CSF).

Endoscopic third ventriculostomy or endoscopic third ventriculocistemostomy (ETV) is an example of a particular endoscopic procedure performed to treat pathological disruption of normal biological fluid flow. ETV is a procedure used for relieving hydrocephalus, a medical condition in which cerebrospinal fluid (CSF) accumulates in the ventricles of the brain due to obstruction of the flow of CSF within or from the ventricles. The accumulation of CSF increases pressure inside the brain, which in turn causes enlargement of the cranium and compression of intracranial brain tissue. Hydrocephalus most frequently occurs in young children, but is also found among adults, and is usually accompanied by neurological deterioration or death.

A standard method to relieve hydrocephalus is to shunt CSF from the brain into the abdominal, venous or peritoneal space. The shunt procedure employs a valved CSF shunt system connected to a plastic drainage line that diverts CSF out of the brain. A specific example of this procedure is ventriculoperitoneal (VP) drainage, which is commonly used to treat hydrocephalus. However, such shunts often fail when they become infected or require surgical revision to relieve obstruction of the shunt. To help avoid such problems, endoscopic third ventriculostomy (ETV) is now commonly used to treat obstructive hydrocephalus, such as that caused by an obstruction of the Aqueduct of Sylvius that communicates between the third and fourth ventricles. ETV creates a surgical fenestration between the third ventricle and the subarachnoid space to permit drainage of excess CSF.

ETV can be performed by placing a burr-hole anterior to the coronal suture of the skull and introducing an endoscope through the brain, into the lateral ventricle and through the foramen of Monro to gain access to the floor of the third ventricle. A fenestration (a ventriculostomy opening) is then surgically created in the floor of the third ventricle, anterior to the basilar artery. The fenestration can be made, for example, by introducing through the floor of the ventricle a blunt guide wire, closed forceps, laser, ultrasonic probe, or the tip of the endoscope itself. The fenestration hole is then enlarged to approximately 5 mm by expanding the tip of a Fogarty balloon catheter in the fenestration or by using an instrument designed for purposeful dilation of the fenestration. One advantage of the ETV procedure is that it does not require an indwelling, permanent shunt catheter that is subject to occlusion or infection.

Although ETV has greatly improved the treatment of hydrocephalus, the ventriculostomy opening sometimes becomes partially or completely occluded as scar tissue forms at the fenestration site. Even in carefully selected patients with obstructive hydrocephalus, technically successful endoscopic third ventriculostomy results in alleviation of hydrocephalus in 60% to 70% of subjects, with up to 40% of subjects having an unsatisfactory clinical outcome. A significant proportion of patients who fail to respond to ETV suffer from secondary closure of the ETV site due to scarring and/or arachnoidal adhesions, and may require subsequent surgical procedures to reestablish patency of the opening or alternatively may result in lifetime ventricular shunt dependency.

This problem with ETV illustrates a more general problem with many endoscopic and other surgical procedures that create artificial openings inside the human body. Surgically created openings in biological interfaces, such as the walls of an organ or other anatomic structures, frequently close as a result of a normal inflammation and healing processes. It would therefore be useful to have a method or device that would maintain the patency of such openings for a sustained period of time.

### BRIEF SUMMARY OF THE DISCLOSURE

The present disclosure provides a method for maintaining patency of an opening through an interface inside the human body by introducing a radially self-expanding hollow stent into the opening through an endoscope that radially compresses the stent. The stent has enlarged ends and a constricted intermediate portion. The shape of the stent allows it to be placed with its constricted intermediate portion situated in the opening while the enlarged ends remain outside of the opening on opposite sides of the opening. The self-expanding stent is allowed to expand in situ such that the enlarged ends inhibit dislodgement of the stent from the opening. A lumen through the stent permits the free flow of fluid through the opening while maintaining patency of the opening.

In particular embodiments, the stent is biodegradable, such that it degrades or otherwise dissolves over time (for example in one to six months). Once the stent has degraded after this period of time, the incidence of scarring or other closure of the opening is reduced. The method can be implemented using an endoscopic surgical procedure for treating hydrocephalus of the brain that increases the success rate of the surgery and reduces the chance of secondary failure. Such a method can include introducing an endoscope into the third ventricle of the brain; fenestrating the floor of the third ventricle to create an opening that fluidly communicates between the third ventricle and subarachnoid space; enlarging the opening; and placing the stent into the opening.

Also disclosed herein is an interfacial stent for maintaining patency of an opening in a biological interface (such as a wall of an organ or substructure thereof, such as a ventricle of a brain) in a human body. The stent includes two enlarged ends and a constricted intermediate portion. The stent is self-expandable, for example being made of a material that has resilient memory, and may be biodegradable. In particular examples, when the two enlarged ends are expanded, each has a diameter substantially greater than a diameter of the constricted intermediate portion that extends through and fills the opening, and/or about the same or greater than the length of the stent. In other examples, the stent has a substantially hollow body defined by an open surface structure that allows flow of the body fluid therethrough.

In one example, the stent is made of bioabsorbable material that degrades in the body over a controlled period of time (such as one to six months). The self-expandable nature of the stent allows it to be introduced into an opening using a compression device (such as a catheter or endoscope lumen) that maintains the stent in a reduced diameter state until it emerges from the device. After emergence, the stent radially expands to permit its secure deployment in the interface opening.

Other features and advantages of the invention will become more readily understandable from the following detailed description and figures.

### BRIEF DESCRIPTION OF THE DRAWINGS

The present description of the stent and its method of use will be described in detail along with the following figures, in which like parts are denoted with like reference numerals or letters.

FIG. 1 is a schematic sagittal section view of the human brain in a child.

FIG. 2 is a schematic top view of a portion of the floor of the third ventricle in the human brain, illustrating a surgical fenestration in the floor of the ventricle.

FIG. 3 is a cross-sectional view of the floor of the third ventricle showing a ventriculostomy hole.

FIG. 4 is a schematic view illustrating placement of the stent through a fenestration in the floor of the ventricle using an endoscope.

FIG. 5 is another schematic view showing deployment of the stent through the fenestration.

FIG. 6 is another schematic view showing radial self-expansion of the stent within the fenestration.

FIG. 7 shows one example of a self-expandable stent in a compressed form as it would be found inside the endoscope prior to deployment.

FIG. 8 shows the stent of FIG. 7 after it has radially expanded.

FIG. 9 shows a second example of a self-expandable stent in a compressed form as it would be found inside the endoscope prior to deployment.

FIG. 10 shows the second exemplary self-expandable stent after it has radially expanded.

### DETAILED DESCRIPTION

A. Terms

In the present description, the terms “opening”, “hole”, “orifice”, “fenestration”, “perforation” and “stoma” all refer to an opening, either naturally existing or artificially created, through an interface of a human body part such as a tissue or membrane. Such interfaces may be found either externally (for example through an ear lobe or other skin surface) or internally (such as the wall of a hollow organ, or wall of a substructure of an organ, such as the ventricles of the brain or the interventricular lumen). In contrast, the term “lumen” refers to the open space within an elongated tubular vessel. Hence an opening, hole, fenestration or perforation is typically present in tissue interface, in contrast to a lumen, which extends through a tubular or elongated extended tissue structure. In addition, the embodiments of the stent disclosed herein are devised to maintain the patency of an artificially created opening, instead of restoring patency of a pre-existing lumen that has become occluded by a pathological process (such as atherosclerosis).

B. Disclosed Embodiments

The disclosed embodiments of the stent are generally designed to maintain patency of an anatomic interface opening (such as a surgically created fenestration) along the entire length of the opening, by retention of the stent within the interface opening by its enlarged ends disposed on opposing faces of the opening. This is distinguished from the prior art use of a stent in a lumen of an elongated tubular vessel such as vascular artery, in which the stent occupies only an intermediate section of an elongated vessel and is contained entirely within the lumen of the elongated vessel.

Several representative embodiments of the stent and methods of its use are disclosed herein for purposes of illustrating how to make and use certain examples of the invention. The representative embodiments are not intended to be limiting in any way.

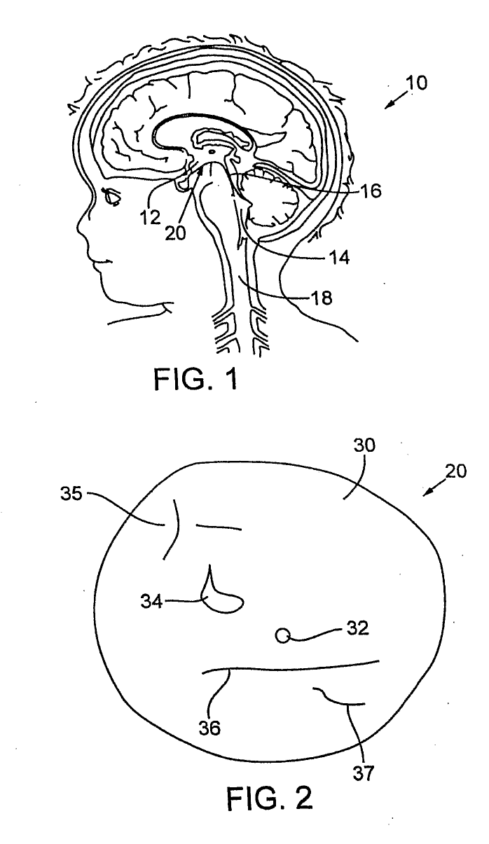

FIG. 1 shows a schematic sagittal section view of human brain 10. Viewable from the sagittal section view is a third ventricle 12, a fourth ventricle 14, and an Aqueduct of Sylvius 16 which in a normal condition communicates between third ventricle 12 and fourth ventricle 14. CSF from fourth ventricle 14 circulates around spinal cord 18 which depends from the brainstem. Also shown in this view is the floor of the third ventricle 20.

A subject who suffers obstructive hydrocephalus often has a blockage of the normal flow of CSF through the ventricular system and the subarachnoid space. For example, a barrier to flow can form within an obstructed Aqueduct of Sylvius 16, which allows abnormal amounts of CSF to accumulate in the proximal portions of the ventricular system, for example in the third ventricle 12 and lateral ventricles. This CSF accumulation is a common cause of hydrocephalus which ultimately causes megalocephaly (enlargement of the head), and compression of neural pathways that leads to deterioration of neurological status, disability and/or death.

FIG. 2 is a schematic top view of wall 30 of the floor of the third ventricle 20 in human brain 10. This view schematically represents what is visible through an endoscope (not shown in FIG. 2) that is introduced into the third ventricle 12 through an endoscopic channel that communicates with a surgical opening through the skull anterior to the coronal suture (not shown). Schematically shown in FIG. 2 are several parts in the brain visible through the semi-transparent floor of the third ventricle 20, including hypophyseal portal veins 35, pituitary gland 34, posterior cerebral artery 36, and posterior perforating arteries 37.

During endoscopic third ventriculostomy (ETV), a fenestration 32 is created in the floor of third ventricle 20 to re-establish flow of cerebrospinal fluid from the third ventricle 12 (FIG. 1) to the subarachnoid space (not shown) underneath the floor of the third ventricle 20. Various methods are known for making this fenestration, including mechanical means, laser and ultrasonic vibration. Usually, fenestration 32 needs to be enlarged after initial formation to achieve a satisfactory size for the purpose of establishing a desired flow of CSF. Enlargement may be performed using a catheter or using an instrument designed for purposeful dilation of fenestrations. The catheter or the dilation instrument may be introduced through a working channel of the endoscope.

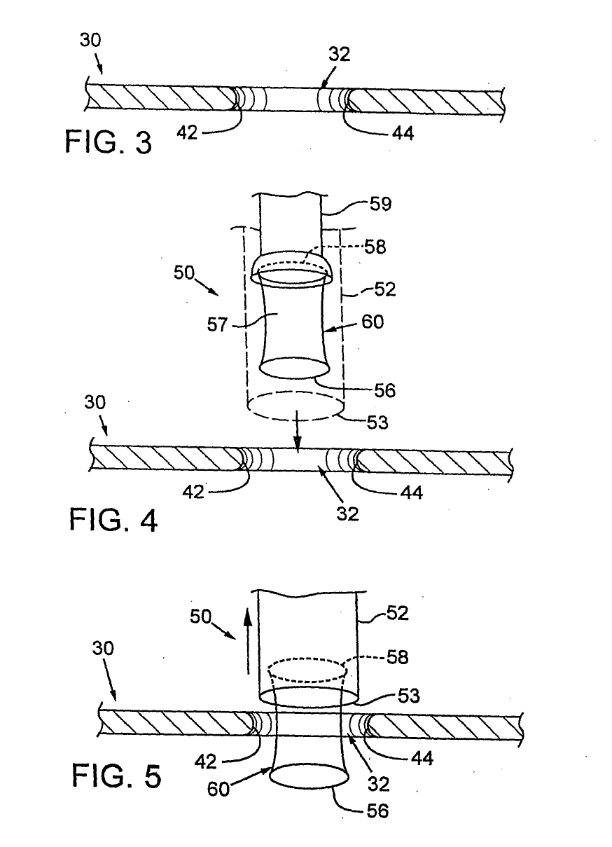

FIG. 3 shows a cross-sectional view of a portion of the floor of third ventricle 30 in which fenestration 32 has been established. Fenestration 32 is defined by perimeter edges 42 and 44. Where fenestration 32 has a circular shape, perimeter edges 42 and 44 are parts of the same continuous inner peripheral edge.

As previously discussed, up to 40% of ETV surgeries do not result in satisfactory resolution of hydrocephalus. A significant proportion of patients who fail to respond to ETV suffer from secondary re-closure of their ETV opening (fenestration 32) due to scarring and/or arachnoidal adhesion. This secondary occlusion of the fenestration can be avoided by use of the embodiments of the stent disclosed herein. The stent is typically an elongated device having resilient memory that allows it to expand from a radially compressed condition in which it is inserted into opening 32 to a radially expanded condition in which it is securely retained within opening 32.

FIGS. 4 and 5 show a stent 60, according to one embodiment, and one specific approach for deploying the stent 60. The illustrated embodiment of stent 60 has a reduced diameter intermediate portion 57 (also referred to herein as a constricted portion) and two relatively enlarged ends 56, 58 that have a greater diameter than the diameter of intermediate portion 57.

As shown in FIG. 4, after fenestration 32 has been created and enlarged, the tip of an endoscope is advanced toward fenestration 32 to deliver stent 60 from a delivery port 50 into fenestration 32. Endoscopic delivery port 50 has an end 53 and sidewall 52 (both shown in phantom in FIG. 4). End 53 of port 50 is open for delivery of stent 60. Sidewall 52 of port 50 confines stent 60 within port 50 and compresses it radially to reduce its diameter. This radial compression force temporarily maintains stent 60 in a narrowed, reduced diameter condition (that may or may not be also elongated compared to the deployed shape of the stent). A retractable release 59 disposed in the delivery port engages the proximal end 58 of stent 60.

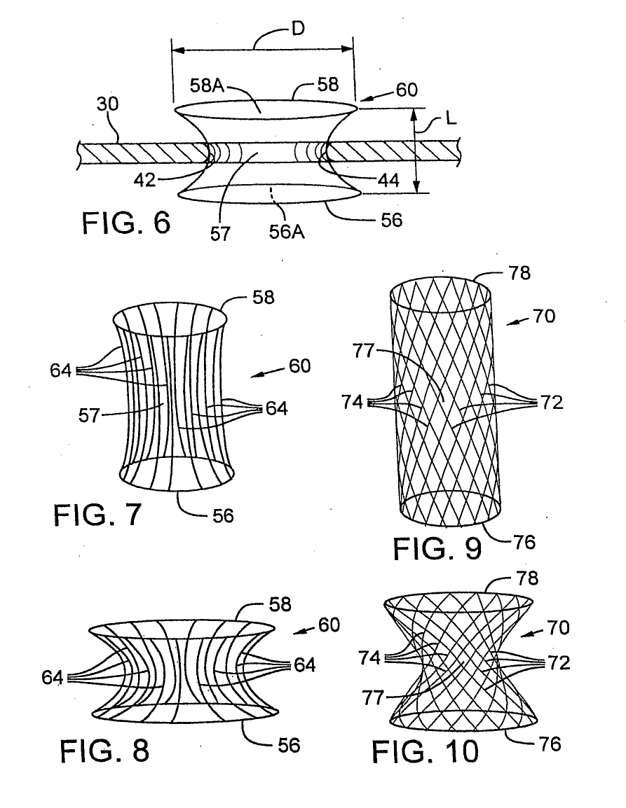

As shown, release device 59 is advanced toward fenestration 32 through the delivery port 59 to deploy stent 60 out of delivery port 50 and place it within fenestration 32, with intermediate portion 57 disposed within fenestration 32 and distal and proximal ends 56, 58 positioned outside fenestration 32 on opposite faces of the wall 30 of third ventricle 20. Once stent 60 has been deployed, and in the absence of external compression, it radially expands as shown in FIG. 6 so that its intermediate portion 57 abuts tightly against the borders of the fenestration, and ends 56, 58 expand to such an extent that stent 60 resists longitudinal dislodgement in either direction out of fenestration 32. For example, radially expanded ends 56 and 58 have a diameter D that is larger than that of constricted intermediate portion 57 and also larger than the diameter of fenestration 32. In the particular embodiment shown, diameter D of enlarged ends 56 and 58 is also at least the same or greater than length L of the expanded stent 60. Length L is measured along the longitudinal axis that is substantially perpendicular to end faces 56A and 58A of ends 56 and 58 of stent 60.

Intermediate portion 57 of radially expanded stent 60 abuts perimeter edges 42 and 44 of fenestration 32 to provide an anatomic barrier to closure of the opening due to inflammatory or other healing processes. However, since stent 60 is hollow and both enlarged ends 56 and 58 are open to fluid flow, retention of stent 60 within fenestration 32 maintains patency of the fenestration 32.

Stent 60 is also preferably made of a bio-compatible material that degrades or otherwise spontaneously dissolves over a controlled or predetermined period of time that is sufficient to inhibit closure of fenestration 32. In many cases, natural inflammatory and healing processes, which initially tend to cause re-closure of the fenestrations, have by this point matured to form a stable and permanent scar tissue around the orifice, thus maintaining rather than occluding the opening. Once the stent has degraded after this period of time, the incidence of scarring or other closure of the opening is reduced.

In a particular example, that period of time is at least one month, for example one to six months. The time required for degrading the stent may be determined based on the observations of a typical interval during which a target opening may be subjected to undesired occlusion. For example, in ETV surgical procedures, the typical failure time during which the ventriculostomy opening may spontaneously close is several weeks. Accordingly, a suitable bioabsorbable material can be selected for making an ETV stent that degrades over several weeks after placement in the brain. For example, a material is chosen that is degraded by the continued flow of CSF through the stent in use. Gradual disappearance of the stent eliminates the necessity of surgical removal of the stent and also reduces the potential risk for infection or other failure that accompanies long term indwelling implants within the body. Furthermore, the bioabsorption time of the interfacial stent may be adjusted based on the selection of the material and/or the construction of the stent (e.g., selecting a mesh or generally solid construction for the stent.)

In particular embodiments of the stent, it would have the following dimensions:

TABLE 1

| | Stent dimensions in | Stent dimensions in |
|---|---|---|
| | compressed state | expanded state |
| Outer diameters of two | 2-5 mm | 4-9 mm |
| ends (56 and 58) | | |
| Length (L) | 3-7 mm | 2-4 mm |
| Outer diameter of | 2-4 mm | 3-7 mm |
| intermediate portion (57) | | |

In one particular embodiment of the stent, outer diameters of ends 56 and 58 are about 3.2 mm when compressed and 6 mm when expanded; length L is 5 mm when compressed and 3 mm when expanded; and outer diameter of intermediate portion 57 (waist) 57 is 3.2 mm when compressed and 5 mm when expanded.

Preferably, stent 60 is introduced into fenestration 32 during the same procedure in which the ventriculostomy fenestration is formed, such that stent 60 is introduced into fenestration 32 immediately after formation of that opening. After stent 60 has been deployed into ventriculostomy opening 32, the endoscopic tools used to introduce the stent into the opening are withdrawn from the body while leaving stent 60 in fenestration 32.

As shown in the above representative example, the present disclosure provides a method and device for inhibiting re-closure of openings in the human body, such as openings through biological interfaces that are designed to establish flow pathways. Re-closure is often caused by natural healing processes in the human body. Such healing processes are particularly effective in infants and young children, who indeed suffer a particularly high failure rate after anatomically successful ETV procedures. Infants and young children represent the majority of patients suffering from newly diagnosed obstructive hydrocephalus and thus would benefit most from the method and the stent of the present disclosure when applied in endoscopic third ventriculostomy.

The application of the method and the stent according to the present disclosure is not limited to ETV procedures. Examples of procedures in which maintenance of patency could be achieved in the disclosed fashion include a variety of cosmetic and therapeutic procedures. Patency of openings for body piercings could be assured, prior to introduction of a metal piercing, by placement of a biodegradable stent (which in this instance would not require a fluid passageway through it). Moreover, there are a number of therapeutic applications, such as maintaining patency of trabeculoplasty, trabeculotomy or sclerotomy openings in the eye for treatment of glaucoma; typanostomy openings in the eardrum for treatment of otitis media; tracheo-esphageal perforation for voice reconstruction after total laryngectomy; tracheostomy openings for establishing a patent airway bypass; openings created in endoscopic nasal and/or facial sinus surgery for maintaining mucous drainage pathways; openings for maintaining bronchopleural fistula for chronic drainage of pleural empyema and other disorders; and openings for the maintenance generally of other intentional permanent or semi-permanent fistulae in biological interfaces.

Although stent delivery has been described in connection with an endoscopic procedure, many other methods are known in the art that may be used to deliver the interfacial stent. In an endoscopic application as shown in the above representative example, existing endoscopic delivery systems may be readily adapted for delivery of the stent. For example, ETV surgery typically utilizes an endoscopic delivery port to deliver a catheter into the newly formed fenestration to enlarge the fenestration. The same endoscopic delivery port may be adapted for delivery of the interfacial stent. Although the stent can be conceivably deployed using a separate delivery port, sharing the same delivery port with the catheter simplifies the system.

In one embodiment, stent 60 is self-expandable, meaning that it expands autonomously when a compression force is removed, without requiring the application of external expansion forces (such as inflation of a balloon within the stent). One example of a self-expandable stent is a stent made of a polymer that has resilient memory, such that the stent expands in a controlled or predetermined fashion to assume a pre-configured shape, usually a shape that the stent had before it was subjected to compressive forces. Additional information about such polymers is provided in a later section of this specification.

Stent 60 also can be bioabsorbable, meaning that the stent will be dissolved or absorbed over time within the human body after a sufficient, usually predetermined period of time to maintain patency of the opening. In the present description, the terms “bioabsorbable”, “bioresorbable” and “biodegradable” have the same meaning and undistinguished from one another despite the awareness that some groups of individuals in the art may regard these terms to have different meanings.

FIGS. 7 and 8 show additional configurations of a self-expanding interfacial stent 60 for placement across an interface of the human body. Stent 60 has opposing ends 56 and 58 which are made of an elastic material forming a ring at each end. A plurality of longitudinal members, or filaments, 64 run substantially parallel to each other between ends 56 and 58 to connect the two ends. Filaments 64 define an inner passageway through which cerebrospinal fluid (or other biological fluid) can flow. Filaments 64 are made of a material having shape memory, as discussed further below, and are formed to at least partially remember a bent or bowed shape. FIG. 7 shows stent 60 in its radially compressed condition in which filaments 64 are axially stretched and ends 56, 58 are spaced at a maximum distance from one another. FIG. 8 shows stent 60 after it has been allowed to expand radially and filaments 64 return to their remembered bowed shape. This radial expansion makes stent 60 shorter, flatter and wider when deployed in fenestration 32.

FIGS. 9 and 10 show yet another embodiment of a self-expanding hollow stent 70 that assumes a tubular configuration in its compressed state, for example conforming to a tubular shape of an endoscope lumen through which stent 70 is introduced into the body. Stent 70 has opposing ends 76 and 78 which are made of an elastic material forming a ring at each end. Two sets of longitudinal members, or filaments, 72 and 74 are used to form an interstitial mesh shaped outer surface that defines the boundary of stent 70. The interstitial mesh shaped outer surface defines a passageway through which cerebrospinal fluid (or other biological fluid) can flow. The two sets of filaments 72 and 74 each run substantially parallel with respect to the filaments in the same set but are slanted in different directions to form an angle between the two sets. Filaments 72 and 74 are made of a material having shape memory, as discussed further below, and formed to at least partially remember a bent shape.

FIG. 9 shows stent 70 in the radially compressed state in which filaments 72 and 74 are axially stretched or elongated. FIG. 10 shows stent 70 after compression forces are removed to allow stent 70 to expand radially as filaments 72 and 74 are allowed to return to their remembered or non-compressed bent shape. This radial expansion makes stent 70 shorter and flatter. Compared to the embodiment in FIGS. 7 and 8, the embodiment according to FIGS. 9 and 10 has a more stable structure. As particularly illustrated in FIGS. 9 and 10, stent 70 has two frustoconical sections, each of which tapers from a respective end 76, 78 to a common intermediate portion 77. The frustoconical sections can be separately formed and subsequently joined to each other at their tapered ends to form the stent. In alternative embodiments, the stent can comprise two tapered frustopyramidal sections, which can be formed in a similar manner. Stent 70 is illustrated as symmetric in shape, having both a transverse and longitudinal axis of symmetry.

Although illustrated in these examples as filamentous, stent 60 may also be made of appropriate sheet- or fabric-like materials with appropriate resilience and memory. The sheet- or fabric-like material may either be in an interstitial mesh pattern (either macroscopically or microscopically), or in a solid shape. The boundary formed by the sheet- or fabric-like material may be either permeable or impermeable to body fluids, as long as stent 60 has an open-ended hollow body that facilitates sufficient body fluid communication through the opening that is intended to be sustained by stent 60.

C. Stent Fabrication

As far as the manufacturing methods are concerned, several types of stents, including metal stents and polymer stents, may be suitable as the trans-interface stent of the present disclosure, with polymer stents being generally more preferable than metal stents.

Polymer Stents

Polymer stents include (but are not limited to) silicone, gelatin film, collagen film or matrix, polysaccharide matrices, and elastomer stents. Compared to metal stents, polymer stents are relatively newer products. One advantage that polymer stents have over metal stents is that they can be bioabsorbable/biodegradable. For this reason, polymer stents are more preferred for the applications disclosed herein.

An ideal stent may have the following characteristics (which are not essential requirements of the invention): (1) inexpensive to manufacture; (2) easy to deploy; (3) sufficiently rigid to resist radial forces; and (4) disappears after treatment without leaving behind harmful residue. Polymer devices that have this capability include resilient collagen materials, resilient gelatin films and biodegradable polymers such as polyesters, polyorthoesters, polyanhydrides, polyglycolic acid and poly(glycerol-sebacate) or PGS. For example, although less flexible, polyglycolic acid tubes provide results equivalent to silicone rubber but are absorbed in seven days and thereby obviate the need for any additional procedure to remove the stent. For applications in which it is desired that the stent have resilient memory, these biodegradable materials can be combined with other polymers that provide elastic recoil to a predetermined shape. A suitable biodegradable polymer available commercially is GELFILM®, an absorbable gelatin film made by Pharmacia & Upjohn (now a division of Pfizer).

Other suitable biodegradable polymers are discussed in U.S. Pat. No. 6,719,934, which patent is incorporated by reference to the extent that it discloses the polymers. These biodegradable polymers include polylactide bioabsorbable polymer filaments, helically wound and interwoven in a braided configuration to form a tube. Polylactide bioabsorbable polymer includes poly(alpha-hydroxy acid) such as poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester, poly(amino acids), or related copolymers materials, each of which have a characteristic degradation rate in the body. For example, PGA and polydioxanone are relatively fast-bioabsorbing materials (weeks to months) and PLA and polycaprolactone are a relatively slow-bioabsorbing material (months to years).

In addition, tyrosine-derived polycarbonate materials developed by Integra LifeSciences Holdings Corp. (Plainsboro, N.J.) may also be suitable for making the interfacial stents of the present disclosure. Another suitable example is bioresorbable, biocompatible and resilient bovine collagen materials developed by Integra LifeSciences Holdings Corp. Such collagen materials have been successfully used for various dental and surgical purposes, but a resilient form of such materials, either in filaments or sheets, may also be a good choice for fabricating the stents of the present disclosure.

A particular example of a biodegradable, self-expandable stent is the L-lactide-glycolic acid co-polymer with a molar ratio of 80:20 (SR-PLGA 80/20). This stent is sold under the product designation SpiroFlow (from Bionx Implants, Ltd., Tampere, Finland) and is disclosed in Laaksovirta et al., J Urol. 2003 August; 170(2 Pt 1):468-71. See also Chepurov et al., Urologiia. 2003 May-June; (3):44-50.

Other bioresorbable polymers under investigation by others may also be suitable. For example, a bioresorbable polymer stent incorporating natural polymers has been described by Bier and coworkers (Bier, J. D., et al., Journal of Interventional Cardiology, 1992. 5(3): p. 187-193.), where type I collagen was formed into a solid tube structure without slotted sides. Bioresorbable microporous intravascular stents were constructed by Ye and colleagues (Ye, Y -W., et al., ASAIO Journal, 1996. 42: p. M823-M827. Ye, Y.-W., et al., Annals of Biomedical Engineering, 1998. 26: p. 398-408.). These stents were extremely porous, and a gradient could be produced from various surfaces of the stent.

As noted, a stent constructed of a bioabsorbable polymer provides certain advantages relative to metal stents such as natural decomposition into non-toxic chemical species over a period, of time. Also, bioabsorbable polymeric stents may be manufactured at relatively low manufacturing costs since vacuum heat treatment and chemical cleaning commonly used in metal stent manufacturing are not required.

In addition, certain materials thought to be unsuitable for intraluminal stents used in vascular applications may be suitable for the stents disclosed herein. Intraluminal stents used in vascular applications have stringent requirements for materials to exhibit strong mechanical properties as structural support and desirable hemodynamics. Due to its distinctive application environment, interfacial stents may not require such stringent mechanical properties for the materials. For example, unlike the endovascular environment, an interfacial environment is less likely to exert high mechanical stress on the stent.

Although multiple examples of embodiments have been disclosed, workers skilled in the art will recognize that changes may be made in form and detail without departing from the spirit and scope of the invention.

1. A method for maintaining patency of an opening inside the human body, comprising:
introducing a radially self-expanding hollow stent into the opening through an endoscope that radially compresses the stent, wherein the stent has enlarged ends and an intermediate portion having a reduced cross-sectional profile, and the stent is introduced into the opening with its intermediate portion extending through the opening and the enlarged ends positioned outside of the opening; and
allowing the self-expanding stent to expand for retention within the opening by the enlarged ends on opposing faces of the opening.

2. The method of claim 1, wherein the stent is bioabsorbable, and degrades over time within the body after a sufficient period of time to maintain patency of the opening.

3. The method of claim 1, further comprising forming the opening by forming a surgical fenestration inside the human body.

4. The method of claim 3, wherein the surgical fenestration is formed in a wall of a ventricle of the brain to establish a path of cerebrospinal fluid flow from the ventricle to a sub-arachnoid space.

5. The method of claim 4, wherein the surgical fenestration is formed in a floor of the third ventricle.

6. The method of claim 3, wherein introducing the radially self-expanding hollow stent into the opening takes place substantially immediately after the fenestration has been artificially created.

7. The method of claim 1, wherein the stent comprises a resilient material that is compressed by delivery through the endoscope, but which expands after delivery from the endoscope into the opening.

8. The method of claim 7, wherein the resilient material comprises L-lactide-glycolic acid co-polymer with a molar ratio of 80:20 (SR-PLGA 80/20), a biocompatible polymer, a biocompatible elastomer, a resilient collagen material, a polysaccharide matrix, or a bioabsorbable gelatin film.

9. The method of claim 1, wherein the intermediate portion is a tapered intermediate portion.

10. The method of claim 9, wherein the stent is symmetric in shape.

11. The method of claim 10, wherein the stent comprises two joined frustoconical sections.

12. The method of claim 1, wherein the stent further comprises a material having shape memory such that the stent is stretchable into an elongated shape along a longitudinal direction and at least partially returns to a remembered shape through expanding along a radial direction.

13. The method of claim 12, wherein the stent comprises multiple longitudinally extending filaments made of the material having shape memory.

14. The method of claim 13, wherein the multiple filaments comprise an interstitial mesh.

15. The stent of claim 1, wherein the enlarged ends of the stent each comprise an elastic material.

16. The method of claim 1, further comprising withdrawing from the opening any surgical instrument used for introducing the stent into the opening to leave the stent in the opening.

17. The method of claim 1, wherein introducing the stent into the opening comprises introducing the stent using an endoscopic surgical procedure.

18. The method of claim 17, wherein using an endoscopic surgical procedure comprises providing a multifunctional telescopic port that is used for sequentially creating the opening and delivering the stent.

19. The method of claim 1, wherein the stent is introduced into the opening through an endoscopic delivery port, the stent being constrained within a tubular portion of the delivery port and held by a retractable release device before being introduced into the opening.

20. The method of claim 1, wherein the intermediate portion contacts an edge of the opening after the stent has expanded.

21. An endoscopic surgical method for treating hydrocephalus of a brain, comprising:
introducing an endoscope into the third ventricle of the brain;
fenestrating the floor of the third ventricle to create an opening fluidly communicating between the third ventricle and a subarachnoid space;
enlarging the opening;
placing a stent into the opening; and
retrieving from the opening any surgical instrument used for placing the stent into the opening to leave the stent in the opening to maintain the patency of the opening.

22. The method of claim 21, wherein the stent comprises a distal portion with a distal end, an intermediate portion, and a proximal portion with a proximal end, and after the stent has been introduced into the opening, the proximal end and the distal end each have a diameter greater than the opening, and the proximal end and the distal end are on two opposing sides of the opening while the intermediate portion passes through the opening.

23. The method of claim 22, wherein the intermediate portion of the stent has a diameter smaller than the diameters of the distal end and the proximal end.

24. The method of claim 23, wherein the stent tapers from the proximal end and the distal end toward the intermediate portion.

25. The method of claim 21, wherein the distal portion and the proximal portion each have a frustoconical or frustopyramidal shape.

26. The method of claim 21, wherein the stent is bioabsorbable.

27. The method of claim 21, wherein the stent is self-expandable.

28. The method of claim 21, wherein placing the stent into the opening further comprises:
delivering the stent through an endoscopic delivery port adjacent the opening, wherein the stent is advanced through the endoscopic delivery port by a retractable delivery device;
releasing the stent;
allowing the stent to expand such that the proximal end and the distal end each expand from a first diameter to a second diameter, wherein the second diameter of the proximal end and the second diameter of the distal end are both greater than the opening.

29. The method of claim 28, wherein the stent is delivered into the opening before it is released by the retractable delivery device.

30. A stent for maintaining patency of an opening at an interface in a human body, the stent comprising:
two enlarged ends; and
an intermediate portion defining a cross-sectional profile that is smaller than that of the enlarged ends;
wherein the stent is biodegradable and expandable;
wherein the stent comprises a substantially hollow body defined by an open surface structure which allows flow of a body fluid through the stent.

31. The stent of claim 30, wherein the stent is self-expandable.

32. The stent of claim 30, wherein the stent is bioabsorbable.

33. The stent of claim 30, wherein the stent tapers from the enlarged ends toward a location intermediate the enlarged ends.

34. The stent of claim 33, wherein the stent is symmetric in shape with respect to an axial direction.

35. The stent of claim 34, wherein the stent comprises two joined frustoconical sections.

36. The stent of claim 30, wherein the open surface structure is an interstitial mesh of filaments.

37. The stent of claim 30, wherein the stent comprises a resilient material.

38. The stent of claim 37, wherein the resilient material comprises L-lactide-glycolic acid co-polymer with a molar ratio of 80:20 (SR-PLGA 80/20), a biocompatible polymer, a biocompatible elastomer, a resilient collagen material, a polysaccharide matrix, or a bioabsorbable gelatin film.

39. The stent of claim 30, wherein the stent comprises a material having shape memory.

40. The stent of claim 30, wherein the enlarged ends comprise an elastic material.

41. The stent of claim 30, wherein the stent comprises multiple filaments extending between the enlarged ends, the filaments being made of a resilient material.

42. The stent of claim 30, wherein when expanded the two enlarged ends have a diameter about the same or greater than a length of the stent.

43. An artificial fluid pathway created in a membrane in a biological body such as a human body to facilitate fluidic communication, comprising:
an artificially created opening in the membrane; and
a hollow stent situated in the opening, wherein the stent has enlarged ends and a constricted intermediate portion, the intermediate portion extending through the opening and the enlarged ends being positioned outside of the opening, and wherein the stent is capable of maintaining the patency of the opening for an extended period of time without support of an additional surgical member.
